(19) United States
(12) Patent Application Publication
(10) Pub. No.: US 2021/0368868 A1
(43) Pub. Date: Dec. 2, 2021

(54) VAPORIZATION DEVICE USING PRESSURE SENSOR FOR AIRFLOW DETERMINATION

(71) Applicant: Michael Trzecieski, Toronto (CA)

(72) Inventor: Michael Trzecieski, Toronto (CA)

(21) Appl. No.: 17/002,413

(22) Filed: Aug. 25, 2020

Related U.S. Application Data
(60) Provisional application No. 62/892,595, filed on Aug. 28, 2019.

(51) Int. Cl. A24F 40/51; A24F 7/00; A24F 40/485; A24F 40/42; A24F 40/10; A24F 40/46; A24F 40/44; A24F 40/57; A61M 11/04; H05B 1/02

(52) U.S. Cl. CPC A24F 40/51; A24F 7/00; A24F 40/485; A24F 40/42; A24F 40/10; A24F 40/46; H05B 2203/021; A24F 40/57; A61M 11/042; H05B 1/0244; A61M 2205/3358; A61M 2205/3368; A61M 2205/3653; A24F 40/44

(57) ABSTRACT

A novel vaporizer device is disclosed having an elongated base extending from a first end to a second end with a mouthpiece formed proximate at the second end of the base, the mouthpiece comprising an inhalation aperture. An air intake manifold is provided with an ambient air input port disposed between a first manifold end and a second manifold end. A fluid path is formed between the first manifold end and the second manifold end the fluid flow path comprising an upstream input port in fluid communication with at least a first absolute barometric pressure sensor and in some embodiments a downstream input port in fluid communication with a second absolute barometric pressure sensor. The absolute pressure sensor for providing an absolute pressure signal to a control circuit for applying power to a heating assembly for heating a vaporizable material in dependence upon the received at least a pressure signal.

This application claims the benefit of U.S. Provisional Application No. 62/892,595 filed on Aug. 28, 2019, the entirety of which is incorporated herein by reference and is a continuation in part of continuation in part of U.S. application Ser. No. 16/207,275 filed on Dec. 3, 2018, the entirety of which is incorporated herein by reference and is a continuation in part of U.S. application Ser. No. 16/801,509 filed on Feb. 26, 2020, the entirety of which is incorporated herein by reference.

### FIELD OF THE INVENTION

This application relates generally to vaporization of phyto materials, and more specifically to vaporizer devices using inhalation airflow sensing.

### INTRODUCTION

The following is intended to introduce the reader to the detailed description that follows and not to define or limit the claimed subject matter.

Phyto materials extracts are used for various therapeutic and health applications. For instance, cannabis extracts may be used to treat a variety of medical conditions, such as glaucoma, epilepsy, dementia, multiple sclerosis, gastrointestinal disorders and many others. Cannabis extracts have also been used for the general management of pain. These cannabis extracts may be filled into cartridges that are known as 510 cartridges that may then contain a heating and vaporizing system and when heated by a heater they are caused to release an aerosol or vapor which then may be inhaled by a user for therapeutic benefits.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with one aspect of this disclosure, which may be used alone or in combination with any other aspect, a vaporizer device is provided comprising: a vaporizer body comprising: an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end, the air intake manifold comprising an ambient air input port disposed between the first manifold end and the second manifold end, the ambient air input port being exposed to an external environment a fluid flow path formed between the first manifold end and the second manifold end the fluid flow path comprising an upstream input port in fluid communication with a first absolute barometric pressure sensor and a downstream input port in fluid communication with a second absolute barometric pressure sensor, each of the pressure sensors for providing of a respective pressure signal to a control circuit, the control circuit for calculating a differential pressure signal from the respective pressure signals; an elongated storage compartment, the storage compartment being configured to store a vaporizable material, the storage compartment comprising an inner storage volume wherein the vaporizable material is storable in the inner storage volume, wherein the inner storage volume is at least partially enclosed by a storage volume housing having a first end and a second end opposite the first end; a heating assembly disposed at the storage volume first end and electrically coupled with the control circuit, the heating assembly comprising a heating element and a wicking element, wherein the heating element thermally coupled to the wicking element, and wherein the wicking element is in fluid communication with the inner storage volume; and a fluid conduit extending through the storage volume housing, the fluid conduit having a fluid conduit inlet at the storage volume first end and a fluid conduit outlet at the storage volume second end, wherein the fluid conduit is in fluid communication with the wicking element, wherein when the inner storage volume is at least partially enclosed by the storage volume housing, the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing through the heating assembly whereby vaporized material is inhalable through the inhalation aperture and power from the control circuit is applied to the heating assembly in dependence upon the differential pressure signal.

In accordance with one aspect of this disclosure, which may be used alone or in combination with any other aspect, a vaporizer device is provided comprising: a vaporizer body comprising: an elongated base extending from a first end to a second end, the elongated base including a pair of opposed sidewalls extending between the first end and the second end and a second end wall at the second end; a mouthpiece formed at the second end of the base, the mouthpiece comprising an inhalation aperture through the second end wall; an air intake manifold mounted to the base, the air intake manifold having a first manifold end and a second manifold end, the air intake manifold comprising an ambient air input port disposed between the first manifold end and the second manifold end, the ambient air input port being exposed to an external environment and formed between the first end and the second end; a fluid flow path formed between the first manifold end and the second manifold end the fluid flow path comprising an upstream input port in fluid communication with a first absolute barometric pressure sensor, the first absolute barometric pressure sensor for providing of a first pressure signal to a control circuit, the first pressure signal being at an other than a first baseline level when air is flowing through the fluid flow path and the first absolute barometric pressure sensor for providing the first pressure signal having the first baseline level to the control circuit when air is other than flowing through the fluid flow path; an elongated storage compartment, the storage compartment being configured to store a vaporizable material, the storage compartment comprising an inner storage volume wherein the vaporizable material is storable in the inner storage volume, wherein the inner storage volume is enclosed by the elongated base and the elongated storage compartment is at least partially enclosed within the vaporized body; a heating assembly electrically coupled with the control circuit, the heating assembly comprising a heating element and a wicking element, wherein the heating element thermally coupled to the wicking element, and wherein the wicking element is in fluid communication with the inner storage volume; and a fluid conduit extending through a portion of the elongated base, the fluid conduit having a fluid conduit inlet and a fluid conduit outlet, wherein the fluid conduit is in fluid communication with the wicking element, the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing through the heating assembly whereby vaporized material is inhalable through the inhalation aperture and power from the control circuit is applied to the heating assembly in dependence upon the first pressure signal being other than at the baseline level.

In accordance with one aspect of this disclosure, which may be used alone or in combination with any other aspect, a vaporizer device is provided comprising: a cartridge comprising: a cartridge housing extending from a distal end of the cartridge to a proximal end of the cartridge; an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing; a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and a fluid conduit extending through the cartridge housing from a conduit inlet at the distal end to a conduit outlet at the proximal end, wherein the fluid conduit is fluidly connected to the heating element assembly, the fluid conduit passes through a center of the heating assembly from the distal end to the proximal end; an inhalation aperture formed at the proximal end of the fluid conduit; a cartridge port having two electrically insulated electrical contacts electrically coupled with the heating element assembly with the fluid conduit propagating through a center thereof; a device body comprising: a cartridge coupling port for electrically coupling to the heating element assembly through first and second electrical contacts and fluidly coupling of the manifold outlet with the fluid conduit distal end; a fluid flow path formed between manifold outlet and an ambient air inlet port, the fluid flow path comprising an upstream input port in fluid communication with a first absolute barometric pressure sensor coupled with a control circuit assembly electrically coupled with the cartridge coupling port, the first absolute barometric pressure sensor for providing of a first pressure signal to a control circuit, the first pressure signal being at an other than a first baseline level when air is flowing through the fluid flow path and the first absolute barometric pressure sensor for providing the first pressure signal having the first baseline level to the control circuit when air is other than flowing through the fluid flow path, the control circuit assembly for controllably providing of pulse width modulated electrical power to the heating assembly in dependence upon the first pressure signal being at an other than a first baseline level and for other than controllably providing of pulse width modulated electrical power to the heating assembly when the first pressure signal is at the first baseline level, wherein when the cartridge is inserted into the vaporization device the cartridge coupling port is coupled with the cartridge port which electrically couples the heating assembly with the control circuit assembly, wherein the storage compartment surrounds the heating assembly and the fluid conduit; and wherein the fluid conduit extends along the entire length of the elongated storage compartment from the distal end to the proximal end.

In some embodiments a vaporizer device is provided wherein the respective pressure signals comprise first and second pressure signals and wherein the first absolute barometric pressure sensor and the second absolute barometric pressure sensor generate a first baseline signal and a second baseline signal when air is other than propagating through the fluid flow path.

In some embodiments a vaporizer device is provided wherein the respective pressure signals comprise first and second pressure signals and wherein the first absolute barometric pressure sensor and the second absolute barometric pressure sensor generate other than a first baseline signal and other than a second baseline signal when air is propagating through the fluid flow path.

In some embodiments a vaporizer device is provided wherein the first and second baseline signals are at other than a same level when air is other than propagating through the fluid flow path.

In some embodiments a vaporizer device is provided wherein the storage volume housing comprises a cartridge housing and the cartridge housing comprises a first cartridge end as the storage volume first end and a second cartridge end as the storage volume second end, wherein the cartridge housing is releasably mounted at least partially within the vaporizer body where the first cartridge end is releasably coupled with the fluid conduit and the electrically coupled with the control circuit.

In some embodiments a vaporizer device is provided wherein the fluid flow path formed between the first manifold end and the second manifold end the fluid flow path comprising a pressure drop element disposed within the fluid flow path between the upstream input port and the downstream input for creating a flow restriction of air flowing through the manifold fluid flow path.

In some embodiments a vaporizer device is provided wherein at least one of the second absolute barometric pressure sensor and the first absolute barometric pressure sensor comprises a temperature sensor.

In some embodiments a vaporizer device is provided wherein the fluid flow path additionally comprising an downstream input port in fluid communication with a second absolute barometric pressure sensor, the second absolute barometric pressure sensor pressure sensors for providing of a second pressure signal to a control circuit, the second pressure signal being at an other than second baseline level when air is flowing through the fluid flow path and the second absolute barometric pressure sensor for providing the second pressure signal having the second baseline level to the control circuit when air is other than flowing through the fluid flow path, wherein power from the control circuit is applied to the heating assembly in dependence upon a difference between the first pressure signal and the second pressure signal and a pulsewidth modulation profile of the power from the control circuit is applied to the heating assembly is varied in relation to the difference.

In some embodiments a vaporizer device is provided wherein the fluid flow path formed between the first manifold end and the second manifold end the fluid flow path comprising a pressure drop element disposed within the fluid flow path between the upstream input port and the downstream input for creating a flow restriction of air flowing through the manifold fluid flow path for creating the signal difference.

In some embodiments a vaporizer device is provided wherein the fluid flow path additionally comprising an downstream input port in fluid communication with a second absolute barometric pressure sensor, the second absolute barometric pressure sensor for providing of a second pressure signal to a control circuit, the second pressure signal being at an other than second baseline level when air is flowing through the fluid flow path and the second absolute barometric pressure sensor for providing the second pressure signal having the second baseline level to the control circuit when air is other than flowing through the fluid flow path, wherein power from the control circuit is applied to the heating assembly in dependence upon a difference between the first pressure signal and the second pressure signal.

### BRIEF DESCRIPTION OF THE DRAWINGS

For a better understanding of the described embodiments and to show more clearly how they may be carried into effect, reference will now be made, by way of example, to the accompanying drawings in which:

FIG. 1A is a top front perspective view of an example of a vaporization device and a cartridge assembly in accordance with an embodiment of the invention;

FIG. 1B is a top front perspective view of the vaporization device base of FIG. 1A with a cartridge assembly removed;

FIG. 1C is a top front perspective view of an insert assembly of the vaporization device of FIG. 1B in accordance with an embodiment;

FIG. 1D is a bottom front perspective view of a cartridge assembly of FIG. 1A in accordance with an embodiment;

FIG. 1E is a cutaway side view of an air intake manifold and cartridge assembly shown in FIG. 1A;

FIG. 1F illustrate an air intake manifold lifted from first and second barometric pressure sensors to expose their sensing ports for clarity;

FIG. 2A illustrates an example of a vaporization device in accordance with an embodiment of the invention;

FIG. 2B illustrates a control circuit assembly positioned within an interior device space;

FIG. 2C illustrates a cutaway view of an air intake manifold and showing first and second pressure sensors;

FIG. 2D illustrates a cartridge inserted into a cartridge receptacle with an inhalation aperture protruding past a housing;

FIG. 2E illustrates a cutaway view of an air intake manifold and showing first and second pressure sensors as well as a cartridge being releasably coupled with a manifold outlet port;

FIG. 2F illustrates a cartridge first and second electrical connections;

FIG. 3A illustrates graphs of a first and second pressure signals and generated by a first and a second absolute pressure sensors as well as a differential pressure signal;

FIG. 3B illustrates a mass airflow standard inhalation profile from an average user;

FIG. 3C illustrates a differential pressure signal of an inhalation of about 400 ml in about three seconds;

FIG. 3D illustrates a differential pressure signal of an inhalation of about 600 mL in about four seconds; and,

FIG. 3E illustrates a differential pressure signal of an inhalation of about 800 ml in about six seconds.

### DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

Embodiments described herein relate generally to vaporization of vaporizable material, such as phyto materials and phyto material products. Although embodiments are described herein in relation to vaporization of phyto material and phyto material products, it will be understood that other vaporizable materials, such as vaporizable nicotine products and/or synthesized vaporizable compounds, or combinations of vaporizable components may be used. For instance, various vaporizable products containing nicotine or plant derived extracts or oils, such as cannabis extract, CBD or terpene extracts and/or synthesized compounds may be used. Phyto material products may be derived from phyto materials such as the leaves or buds of cannabis plants.

Various methods of vaporizing phyto materials and phyto material products, such as cannabis products, are known. Phyto material is often vaporized by heating the phyto material to a predetermined vaporization temperature. The emitted phyto material vapor may then be inhaled by a user for therapeutic purposes.

Devices that vaporize phyto materials are generally known as vaporizers. In some cases, oils or extracts derived or extracted from the phyto materials may also be vaporized. For cannabis oils or extracts, temperatures in the range of about 450 to 800 degrees Fahrenheit may be applied to vaporize these phyto material products may generate phyto material vapor.

The phyto material vapor may be emitted at a temperature that is uncomfortable for a user to inhale. Accordingly, it may be desirable to cool the vapor prior to inhalation.

Phyto material products, such as oils and extracts, may be generated in batches. The batches may be mixed in a liquid or semi-liquid state. This may facilitate testing of the potency of the phyto material product and provide greater consistency of potency throughout a batch of phyto material product.

Phyto material products, such as oils and extracts may be provided in various liquid, semi-liquid forms. These liquid phyto material products may be stored in a cartridge such as a 510 cartridge that may be used with a vaporizer device.

In some cases, a vaporizable material may be added into a cartridge, and in turn, this cartridge is inserted into a vaporizer. However, it may be quite difficult to fill the cartridges with vaporizable material. Typically, a thin syringe is used to inject very dense oil through a very small applicator tip/orifice into the cartridge.

Vaporization devices that provide for removable cartridges to be vaporized may allow users to adjust the type and/or potency of phyto material products being consumed. A user may insert a cartridge of a particular type into their vaporization device based on the desired therapeutic effect. If a different effect is desired, or the cartridge is spent, the old cartridge may be removed and a new or different cartridge may be inserted for subsequent vaporization.

Vaporization of material from a phyto material cartridge may involve airflow through the phyto material cartridge. However, it may be difficult to ensure consistent airflow through the cartridge as the space available within the vaporization devices limits the space available for a fluid conduit through the cartridge and in some cases measuring of the airflow through the cartridge or vaporization device may be preferable.

Embodiments described herein related generally to methods and devices for vaporizing phyto material, in particular liquids containing phyto material such as medical cannabis. In embodiments discussed herein, examples of vaporization devices or vaporizer devices are described that may be used to vaporize cartridges containing vaporizable products such as liquid phyto material products. The example vaporizer devices may be associated with any suitable type of cartridge containing vaporizable liquid materials that is engageable with the vaporizer devices, such as the example cartridges described herein.

Similarly, in embodiments discussed herein, examples of cartridges that are of a particular type that are usable to store liquid vaporizable materials that are vaporizable using vaporizer devices are described.

Similarly, in embodiments discussed herein, examples of devices that contain reservoirs for storing of liquid vaporizable materials that are of a particular type that are usable to store liquid vaporizable materials that are vaporizable using vaporizer devices are described.

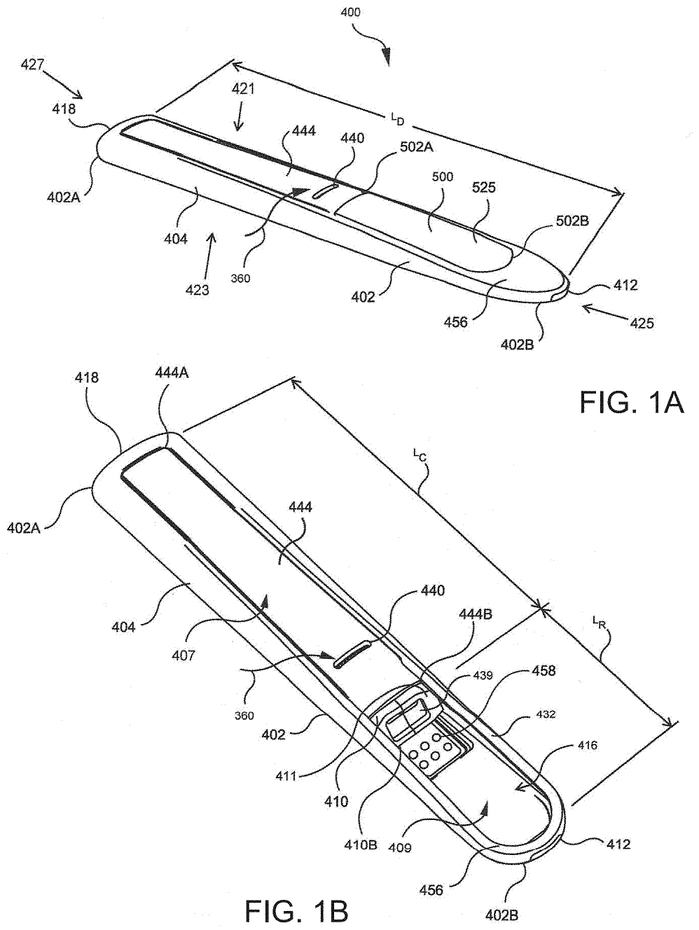

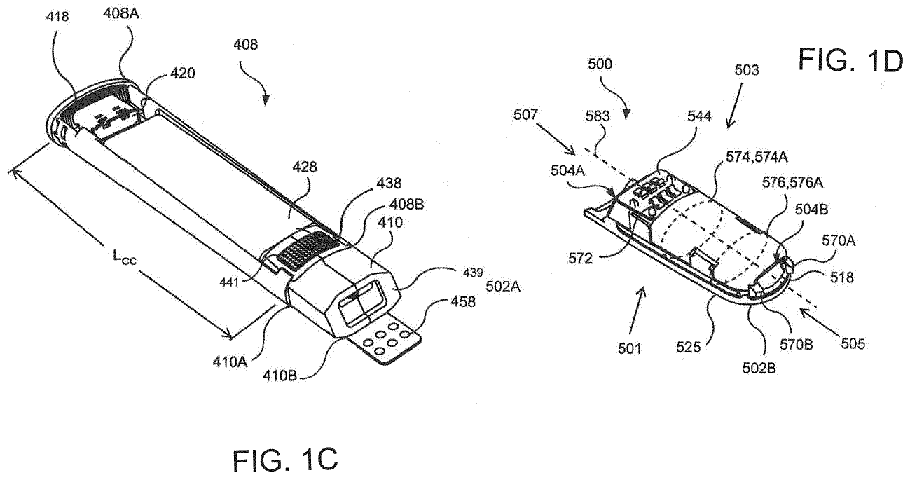

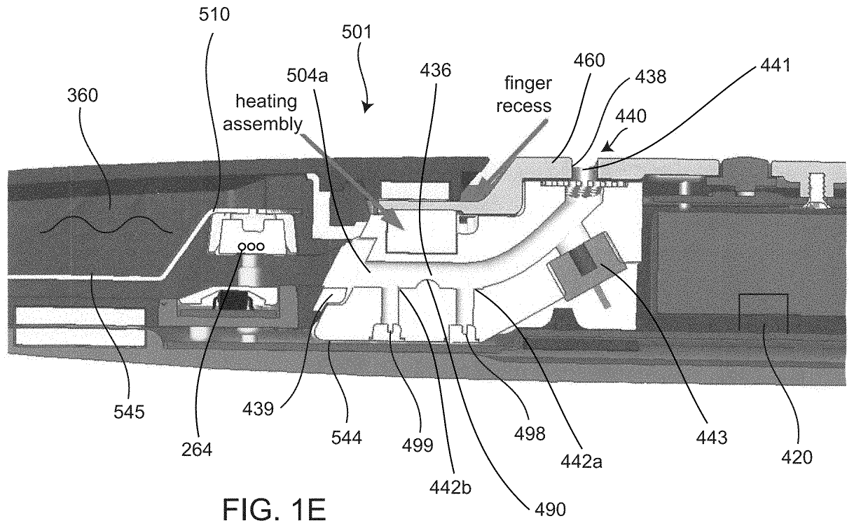

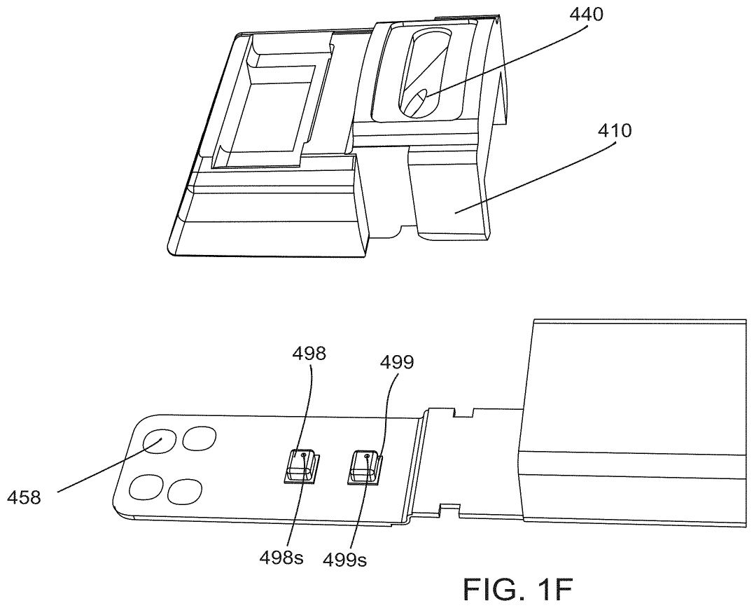

Referring now to FIGS. 1A through 1F, there is shown an example of a vaporization device 400. Vaporization device 400 is an example of a vaporization device usable to vaporize vaporizable material. Vaporization device 400 may be used to vaporize vaporizable material that is provided in a semi-liquid and/or liquid form. In some cases, vaporization device 400 may allow vaporizable materials to be inserted and/or stored in a solid or semi-solid form and subsequently vaporized in a semi-liquid or liquid form.

Vaporization device 400 will be described in combination with an example of a cartridge assembly 500. Cartridge assembly 500 is an example of a cartridge assembly that may be used to store vaporizable material for use with vaporization device 400.

The vaporizer device 400 may have a top side 421, a bottom side 423, a front side 425, a rear side 427 and a pair of opposed lateral sides. As shown, vaporization device 400 includes a device body 402 and a removable cartridge 500. In some embodiments the cartridge maybe integral and non-removable within the vaporizer device 400.

The device body 402 can include a base 404 and a cover 444. The device base 404 may include a plurality of device sections. A first device section 407, proximate the first end 402A, can contain various components of the vaporization device such as a control assembly and/or energy storage member. A second device section 409, proximate the second end 402B can define a receptacle 416 for the cartridge assembly 500 (FIG. 1D).

The base 404 of vaporizer 400 can define a recess 406. In vaporizer 400, the recess 406 extends generally from the first end 402A of body 402 to the second end 402B of body 402. In some cases, the base 404 may be open at the first end 402A. A control assembly 408 can be inserted into the first section 407 of base 404. The control assembly 408 can include a first end closure member 418 that encloses the first end 402A. The closure member 418 may also have an outer rim or lip that may help secure the cover 444 to base 404.

The control assembly 408 may be secured within the base 404, e.g. by frictional engagement with an inner surface 432 of base 404. As with base 102, the inner surface 432 of base 404 may be lined to provide a compressible material that allows the control assembly 408 to be inserted therein with a frictional fit. For instance, the control assembly 408 may be slid into the base 404 initially from the first end 402A. The control assembly 408 may also be further secured to base 404 using fasteners such as screws, bolts, and/or adhesives for example. In some embodiments the control assembly 408 can be secured in place by the cover 444. The cover 444 may be secured to control assembly 408 and/or base 404 using a specialized mechanical fastening. A specialized tool corresponding to the fastening may be used to couple and uncoupled the cover 444 from control assembly 408 and/or base 404.

The base 402 may also have a tapered structure. The base 402 may have a larger cross-sectional area 452 proximate the first end 402A than the cross-sectional area 454 proximate the second end 4026. The first section of the vaporizer 400, with a larger cross-sectional area, may provide recess 406 with an enlarged space within which to store components of the vaporizer such as the control assembly 408 and energy storage members 428. The reduced cross-sectional area of vaporizer 400 proximate the second end 4026, may allow device 400 to provide an inhalation aperture 412 with a size that is more approachable for a user to partially insert into their lips for inhalation.

The control assembly 408 can include a control circuit 420 and one or more energy storage members 428. The control assembly 408 may also include various components generally similar to the first recess section of vaporization device 100, such as the control circuit 420, wireless communication modules 422, 424, 426, energy storage members 428, feedback indicators 430 and so forth.

As shown in FIG. 1C, the air intake manifold 410 in vaporizer 400 can be provided with the control assembly 408. The control assembly 408 can also include a plurality of electrical contacts 458 that are positioned at the second end 4106 of air intake manifold 410. In the example shown, the device electrical contacts 458 extend beyond the second manifold end 458B towards the second end 402B of vaporizer 400. As shown, the device electrical contacts 458 are positioned on a bottom surface of receptacle 416 facing upwards into receptacle 416.

The contacts 458 can be positioned to engage corresponding electrical contacts on the cartridge assembly 500 when inserted into receptacle 416. The electrical contacts 458 may allow for various signals to be transferred between the vaporizer control assembly 408 and the cartridge assembly 500, such as power signals, sensor signals, control signals and the like.

The vaporizer device 400 can also include a cover 444 that can be used to enclose the first section of the vaporizer base 404. FIGS. 1A and 1B show the vaporization device 400 with the cover 444 connected to base 404.

The cover 444 can protect the components of the control assembly 408 from concussive damage and exposure to dirt or debris. As with cover 144, the cover 444 may be manufactured using a non-conductive material to facilitate wireless communication by the control assembly 408. In some cases, the main body of cover 444 may be manufactured using metallic materials that may interfere with signal transmission. In such cases, the end closure member 418 of control assembly 408 may be formed using a non-conductive material, such as plastic, to facilitate signal transmission therethrough.

In some embodiments, the cover 444 may be manufactured using materials having a higher coefficient of friction from base 404. This may provide a user with a different hand feel when grasping device 400. In some cases, the cover 444 may be electrically insulated from the base 404 when secured to base 404. This may facilitate conductive sensing by the control assembly 408, as a user's hand grasping the vaporizer 400 may be detected via capacitive sensing (as the user's hand can couple the base 402 to the cover 444). The control assembly 408 may use these capacitive sensing signals (the base 402 being electrically insulated from the cover 444) to activate the control circuit 420 from a low-power mode to a more active mode in anticipation of user inhalation.

A center of gravity 474 of vaporizer device 400 may be positioned closer to the first end 402A than to the second end 402B of the device 400. The heavier components of vaporizer 400, such as the energy storage members 428, can be positioned within the first device section 407. By providing the majority of the weight of vaporizer device 400 nearer to the first end 402A, the vaporizer device 400 will provide a user with a balanced weight when grasped near the first end 402A. As the inhalation aperture 412 is positioned proximate the second end 402B, a user may be inclined to grasp the vaporizer device 400 around the first section 407 so that the second end 402B can be raised to contact the user's lips and mouth for inhalation.

For instance, the base 404 may be formed as a unitary construction. The base 404 may be manufactured using metal, thermoplastic or ceramic materials such as zirconium oxide or other ceramics. When the base 404 is manufactured using metal, machining processes or metal injection molding processes may be used.

The vaporizer 400 can include a mouthpiece having an inhalation aperture 412 at the second end 402B. The inhalation aperture 412 may be formed as a void section in the second end 402B. Optionally, a removable mouthpiece cover may also be provided with aperture 412.

The base 404 can also define a receptacle 416 configured to receive the cartridge assembly 500. The receptacle 416 may be defined in the second portion 409 of the device base 402 proximate the second end 4026. The receptacle 416 may be formed as a recess within the base 402 into which the cartridge assembly 500 can be inserted.

The inhalation aperture 412 can be fluidly connected to the cartridge receptacle 416. When the cartridge assembly 500 is inserted into the receptacle 416, the inhalation aperture 412 can be fluidly connected to a fluid conduit 504 that extends through cartridge assembly 500 from a cartridge conduit inlet 504A to a cartridge conduit outlet 504B. In some cases, a downstream end 518 of the fluid conduit 504 may extend outward through the mouthpiece to define a protruding inhalation aperture 412. In other cases, the inhalation aperture 412 may be flush with the second end 402B of the device body 402, e.g. as shown.

As with vaporizer 100, the vaporizer 400 can also include an air intake manifold 410. The air intake manifold 410 can be configured to allow ambient air to be drawn into vaporizer device 400 and directed into a cartridge 500 positioned within the cartridge receptacle 416. The air intake manifold 410 can be positioned within a third, central section 411 of the device body 402. In vaporizer device 400, unlike vaporizer 100, the cover 444 extends over the air intake manifold 410 as well as the control assembly 408. As shown, the cover 444 may include an ambient air aperture 440 that can be fluidly coupled to an ambient air inlet 438 of air intake manifold 410. A screen or filter 441 may optionally be positioned at the ambient air inlet 438 to filter ambient air entering the air intake manifold 410 (see e.g. FIG. 1C).

Air intake manifold 410 can extend from a first manifold end 410A to a second manifold end 4108. The first manifold end 410A can be positioned within the recess 406 adjacent to, or contacting, the second end 408B of the control assembly 408. As with air intake manifold 110, the air intake manifold 410 may be mounted to support member 414 and/or positioned adjacent a front end of the support member 414. The second manifold end 4108 can face into the cartridge receptacle 416. A manifold outlet 439 can be positioned at the second manifold end 4108. A manifold fluid flow path 436 may extend between the ambient air inlet 438 and the manifold outlet 439.

The air intake manifold 410 may include a fluid flow sensor 442. The fluid flow sensor 442 can be used to identify ambient air 360 being drawn into the vaporizer 400 via ambient air inlet 438. In some cases, the fluid flow sensor 442 may be configured to identify the volume or mass of air being drawn into the vaporizer 400. The fluid flow sensor 442 can provide flow signals to control circuit 420, to allow control circuit 420 to activate/deactivate the cartridge heating assembly 510 and/or adjust the temperature of the heating element 564.

In the example shown for the intake manifold there is at least an upstream input port 442a and a downstream input port 442b in fluid communication with a manifold fluid flow path 436. A pressure sensing element may be disposed at the at least one of the upstream port 442a and the downstream port 442b. Each pressure sensing element can determine an absolute pressure at the at least the upstream port 442a and the downstream port 442b.

FIG. 1E shows a cutaway side view of an air intake manifold and cartridge assembly shown in FIG. 1A. Referring to FIGS. 1F and 1E, a first absolute barometric pressure sensor 498 is fluidly coupled with its sensing port 498s with the upstream port 442a and a second absolute barometric pressure sensor 499 is fluidly coupled with its sensing port 499s with the downstream port 442b. Where the first absolute barometric pressure sensor 498 and a second absolute barometric pressure sensor 499 provide a first and second pressure signals. Each signal is processed by a control circuit 420. Disposed between the upstream input port 442a and a downstream input port 442b there may be a pressure drop element 490, such as a raised protrusion or another form of obstruction that provides for a flow restriction of air flowing through the manifold fluid flow path 436. For FIG. 1F, the air intake manifold 410 is shown uncoupled from the absolute barometric pressure sensors for clarity.

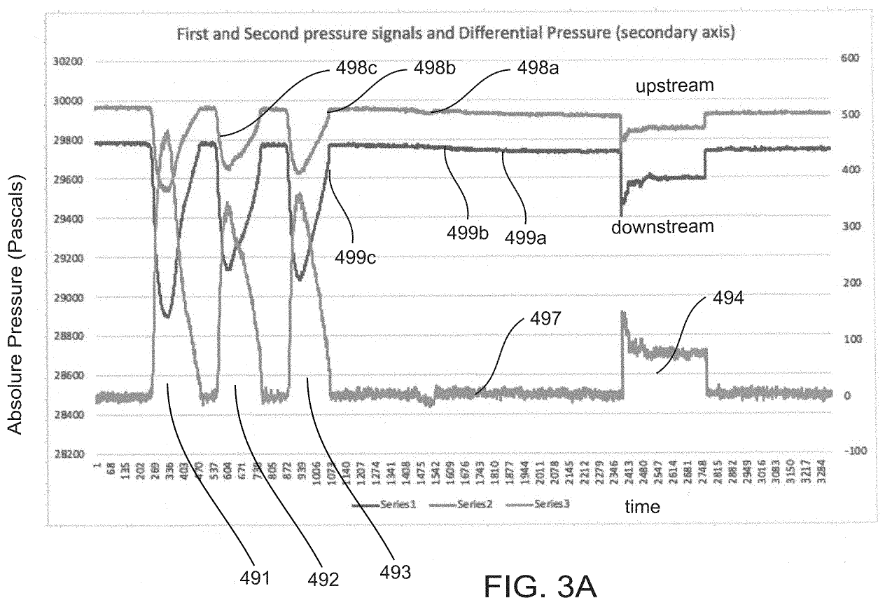

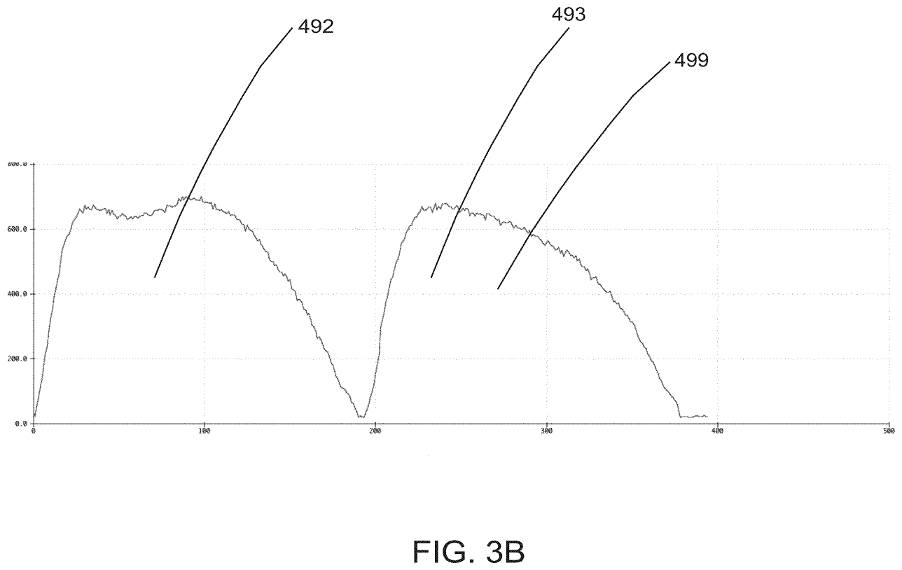

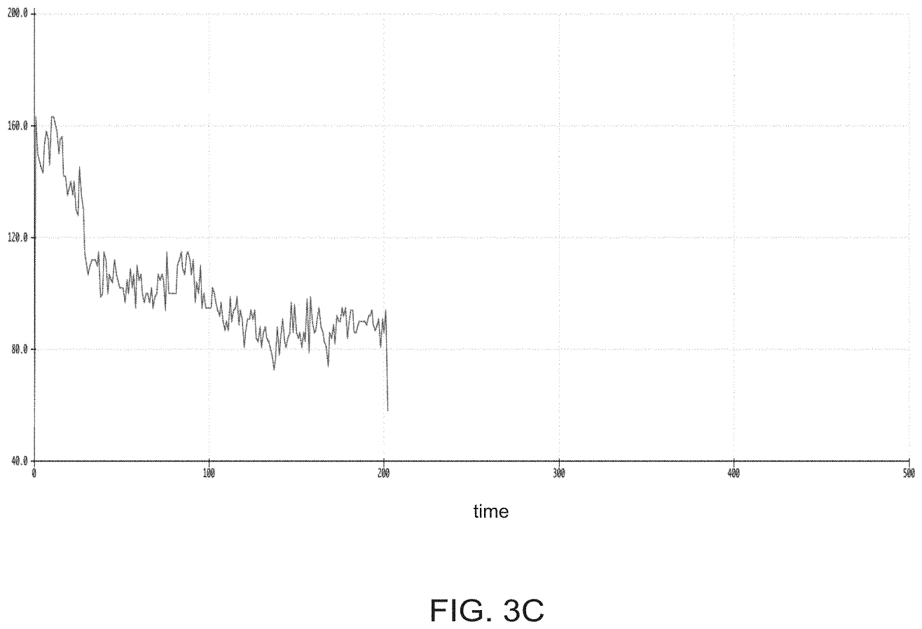

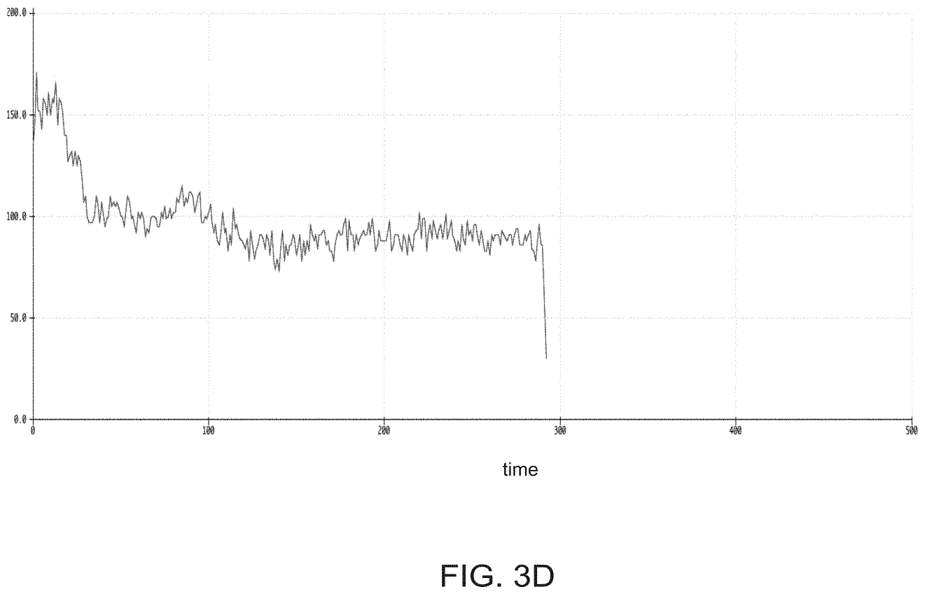

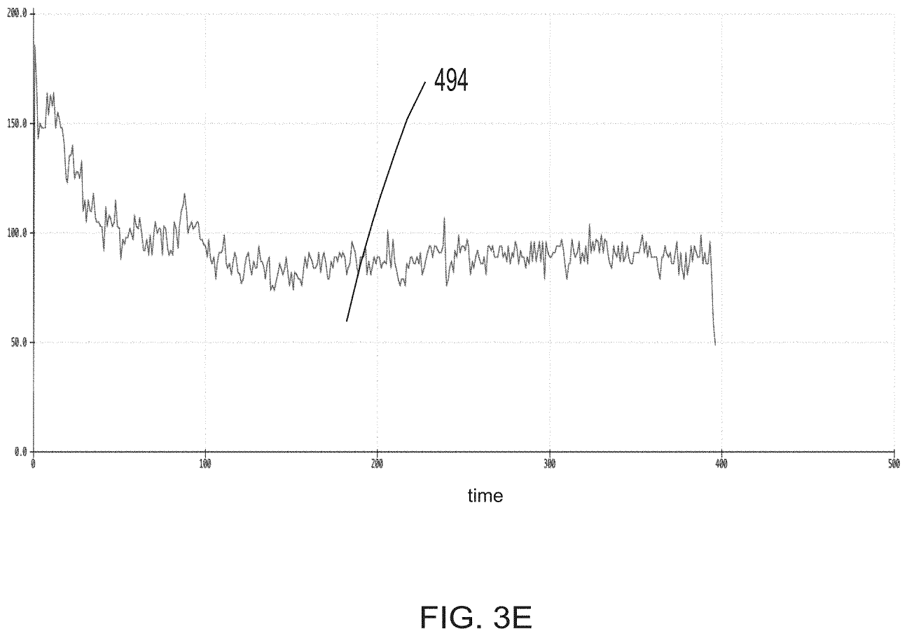

Referring to FIG. 3A and FIG. 1E, in some embodiments when a single absolute barometric pressure sensor 498 is utilized, a baseline level 498b may be obtained from the sensor as the first pressure signal 498a and this baseline level may be indicative of an absolute barometric pressure being sensed at, for example the upstream port 442a. The first pressure may be at an other than baseline level when air is flowing through the fluid flow path and the first absolute barometric pressure sensor for providing the first pressure signal having a baseline level provided to the control circuit when air is other than flowing through the fluid flow path, as is described hereinbelow with reference to FIG. 3A.

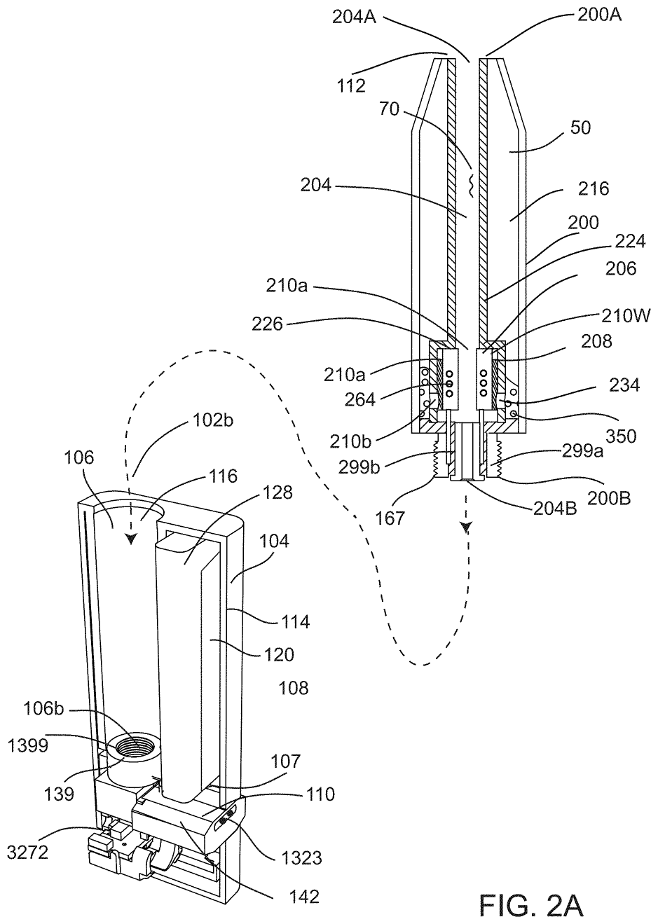

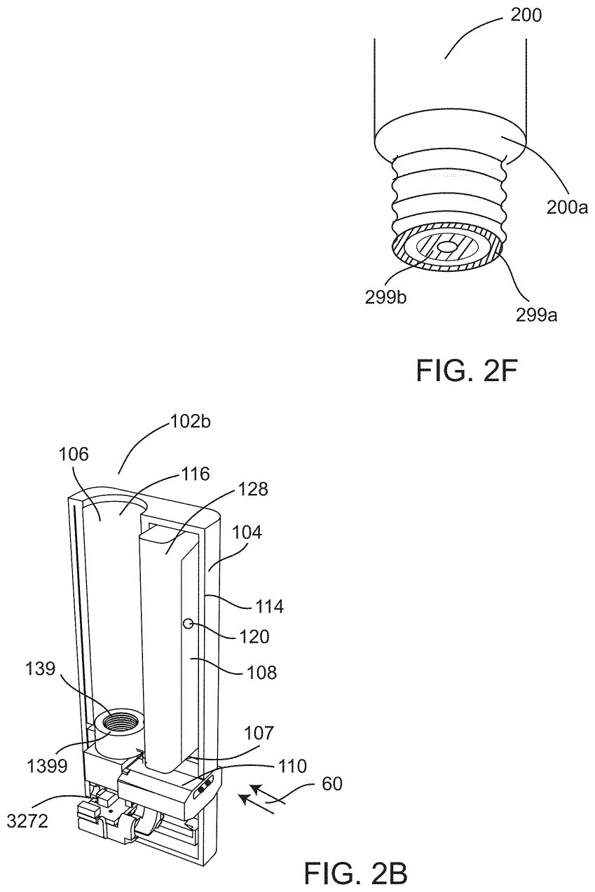

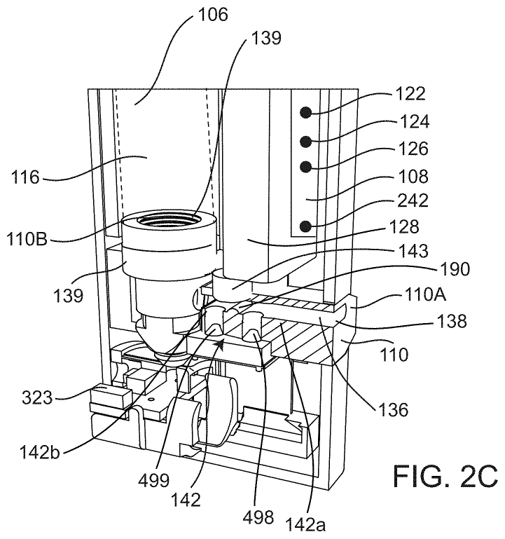

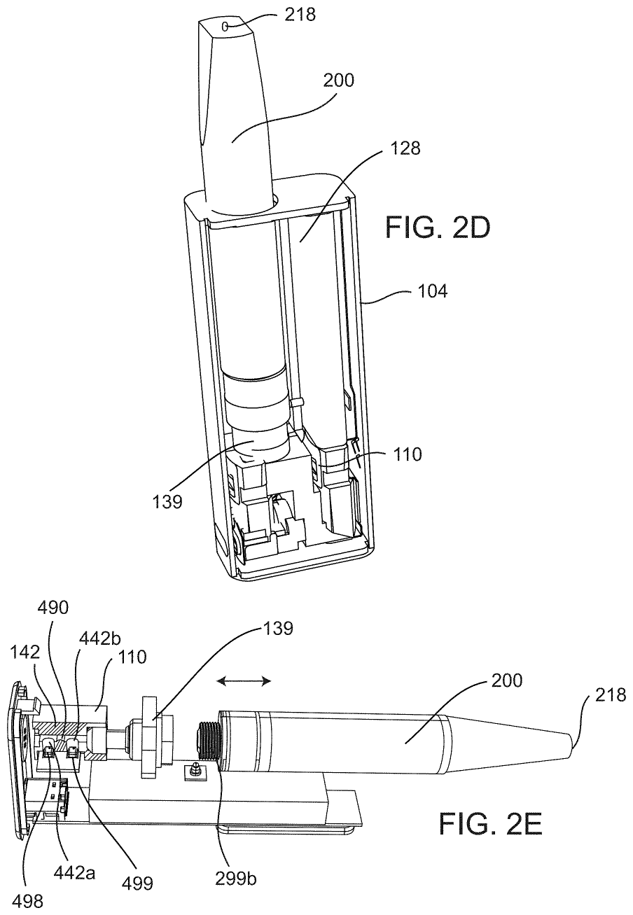

Referring now to FIGS. 2A through 2E, shown therein is an example of a vaporization device 100. Vaporization device 100 is an example of a vaporization device that can be used to vaporize material that may be derived from or contain extracts from phyto materials such as cannabis that maybe stored in a cylindrical storage vessel, such as a 510 threaded cartridge as is well known in the art. Vaporization device 100 may be used to vaporize phyto material products in a liquid or semi-liquid form, which may be referred to herein as vaporizable liquids or liquid vaporizable materials.

Referring now to FIG. 2A shown therein is an example of the vaporization device 100 in accordance with an embodiment of the invention and a vaporizer cartridge for being inserted into a battery unit. The device body 102 and the cartridge 200 are both shown in cutaway views, device body 102 may be used to house and retain various components of the vaporization device 100, such as a control circuit assembly 108, air intake manifold 110, and a cartridge assembly 200.

The vaporization device 100 that may include a device body 102 that may be formed from two housing parts that include a base 104 and a cover 144. The device body 102 may include a top side or proximal side 121, a bottom side or distal side 123, a front side 125, a rear side 127, and opposed lateral sides 129. Vaporization device 100 generally includes, the front side 125. Base 104 defines opposed lateral sides and the rear side 127 and the bottom side of vaporization device 100. The vaporization device 100 may be used to vaporize material that may be derived from or contain extracts from phyto materials such as extracts derived from cannabis when used with the cartridge 200 inserted therein that may be used to store liquid vaporizable material. Vaporization device 100 may be used to vaporize phyto material products in a liquid or semi-liquid form, which may be referred to herein as vaporizable liquids or liquid vaporizable materials.

A cartridge receptacle 106 or receptacle may be defined within the device body 102 and more specifically within at least one of the base 104 and the cover 144. The cartridge receptacle 106 may be shaped to receive and engage a cartridge 200, such as cartridge 200, which is well known in the art. The cartridge receptacle 106 may extend from the proximal side 121 distally towards the distal side 123 and may not protrude past the distal side 123. In FIG. 1B, the cartridge 200 is shown as not being inserted into the cartridge receptacle 106 for clarity. Typically, such a cartridge is about under 11 mm in diameter, and in some cases about 10.2 mm in diameter and may have a length of about 60 mm to 70 mm, details of which will be explained further below.

The receptacle 106 may be defined in the device body 102 may include a portion or section that defines a cartridge receptacle 106 where each of the cover 144 and the base 104 may include the cartridge receptacle 106. In the example shown, the cartridge receptacle 106 is defined by the cartridge receptacle 106 that extend from a base proximal end towards the base distal end device body 102. The cartridge receptacle 106 may be shaped to receive a phyto material cartridge such as cartridge 200 where the cartridge 200 may be in the shape of an elongated cylinder and having a central axis that is approximately parallel with a long axis of the cartridge receptacle 106 within the device body 102 and for cartridge 200 to be sliding along this long axis of the when the cartridge 200 is being inserted into the cartridge receptacle 106 or removed therefrom. The cartridge receptacle having a proximal end 106a for receiving the cartridge, the cartridge for being inserted into the cartridge receptacle from the proximal end 106a to a distal end 106b thereof wherein the cartridge coupling port is distally disposed within the cartridge receptacle 106. The cartridge 200 may be used to store liquid vaporizable material. The cartridge 200 may be removably mounted to the device body 102 within the cartridge receptacle 106 and frictionally or magnetically held therein to facilitate airflow and electrical contact.

The cartridge 200 may include a heating chamber 206 and a storage compartment 216. A storage interface member 224 may include at least one or a plurality of apertures positioned facing the storage compartment to allow vaporizable material to contact a wicking element 208 for flowing into the heating chamber 206. The cartridge may include a proximal end 200A and a distal end 200B opposite the proximal end 200A. An inhalation aperture 112 may be formed at the proximal end 200A of the cartridge 200. Cartridge housing 202 may extend between a cartridge proximal end 200A and a cartridge distal end 200B opposite the cartridge proximal end 202A. A housing sidewall may extend between the cartridge proximal end 200A and the cartridge distal end 200B.

The fluid conduit may extend through the cartridge housing 202 from the cartridge proximal end 200A to the cartridge distal end 200B. The fluid conduit 204 may include a distal end 204A or upstream inlet at the cartridge distal end 200B and it may also include a cartridge conduit outlet or inhalation aperture 112 downstream and proximally disposed from the distal end 204B or inlet and have a conduit proximal end 204A and proximate the cartridge proximal end 200A.

The storage compartment or reservoir may be used to store vaporizable material for use with a vaporizer 100. The storage compartment may be enclosed by the outer housing sidewall 214. In the example shown, the storage compartment may be parallel to the fluid conduit. That is, the fluid conduit 204 may define a passage that extends parallel to the storage compartment 216 and the fluid conduit 204 may be fluidly and thermally coupled to the heating element assembly 210. The storage compartment and the fluid conduit 204 may be concentrically disposed about a central axis of the conduit 204. In some embodiments the storage compartment may be fillable through an elastomeric seal that is punctured by a filling needle that is inserted into the elongated storage compartment for injecting of the vaporizable material therein.

When the cartridge 200 is inserted into the base 104 and more specifically the cartridge receptacle 106 is inserted into the base, the inhalation aperture 112 may protrude past the housing as shown in FIG. 2D. The storage compartment 216 may also be fluidly connected to a heating element assembly 210. The heating assembly may be used to vaporize vaporizable material 350 stored in the storage compartment 216, where the vaporizable material 350 may be drawn from storage compartment 216 and into wicking element 208 that is thermally connected to the heating element assembly 210. Electrical current from an external energy storage member 128 that is external to the cartridge 200 may be directed through heating element assembly 210 when a cartridge coupling port 1399, disposed within the cartridge receptacle 106, is coupled with the cartridge port 167 for electrically coupling to the heating element assembly 210 through first and second electrical contacts and fluidly coupling of the manifold outlet port 139 with the fluid conduit distal end 204B when the cartridge 200 is inserted into the cartridge receptacle 106. The heat emitted by resistive heating element 264 may heat the vaporizable material that is wicked into the heating element assembly 210 to a predetermined vaporization temperature. The heating element assembly 210 may be disposed proximal the distal end of the cartridge 200.

The heating element assembly 210 may also be used with the wicking element 208. The wicking element 208 may at least partially surround the heating element assembly 210. The wicking element 208 may also be arranged coaxially about the heating element assembly 210 and a distal portion of the storage interface member 224 may be oriented coaxially with the heating element assembly 210.

The heating element assembly 210 may be held in place by the storage interface member 224 against the wicking element 208 which is exposed to the vaporizable material from the storage compartment 216 may be drawn to the heating element assembly 210 by wicking element 208. The vaporizable material in the wicking element 208 may then be heated by the heat emitted by a resistive wire that may be embedded within the heating element assembly 210. The storage interface member 224 may surround the heating element assembly 210, and the storage interface member 224 includes a plurality of circumferentially spaced fluid apertures 234 fluidly connecting the heating element assembly 210 to the inner storage volume 216. The heating element assembly 210 and wicking element 208 may be manufactured using a resistive wire embedded in a porous ceramic material. For example, heating element assembly 210 may be manufactured using a porous ceramic and the porous ceramic acts as the wicking element and may obviate a need for a separate wicking element 208.

The heating element assembly 210 may include the resistive heating element 264, which may be in the form of a plurality of resistive heating wire bands may be positioned between the first and second element ends 210A and 2108, e.g. as shown. The resistive bands 264 may be enclosed with an outer wall 210w of the heating element assembly 210. An outer wall of the heating element assembly 210 may be manufactured from a material having limited thermal conductivity, such as a porous ceramic material. The porous ceramic material may initially provide a partial thermal and electrical insulator that allows the resistive heating element 264 to heat up relatively fast due to the low thermal inertia of the heating element assembly 210. The plurality of resistive heating wire bands 264 may be in the form of a coiled wire embedded within the porous ceramic heating element assembly 230. In some cases, the wicking element 208 may be formed integrally with the heating element assembly 210. For example, the heating element assembly 210 may be manufactured from a porous material (e.g. porous ceramics) with pores sized to receive the vaporizable material 350. The pores may also allow the emitted vapor to pass therethrough when resistive heating element 264 is energized, where in some embodiments a 40-50% open porosity with a tortuous pore structure with a pore size ranging from 20 to 90 micron. A resistance of resistive heating element 264 may be about 0.9 Ohms to about 1.7 Ohms.

In embodiments where both heating element assembly 210 and wicking element 208 may be manufactured using porous materials, the pore sizes of the heating element assembly 210 and wick 208 may differ. For instance, the wicking element 208 may have pores with a smaller diameter than the pores of heating element assembly 210. For example, a porous ceramic material used with heating element assembly 210 may be macro-porous having pores with a diameter larger than 50-80 microns. The wicking element 208 may have pores with diameters smaller than 50 microns.

When assembled, the wicking element 208 and the heating element assembly 210 may be positioned concentrically about the heating chamber cavity 226. The heating chamber cavity 226 may be fluidly connected with a fluid conduit 204. Vapor emitted from heating the vaporizable material in wick 208 may then be drawn into fluid conduit 204 through plurality of circumferentially spaced fluid apertures 234 formed within the interface member 224. The fluid conduit 204 propagating from the distal 200B to the proximal 200A ends of the cartridge 200.

Heating element assembly 210 may be positioned within the heating chamber cavity 226 with the wicking element 208 fluidly coupling the fluid conduit 204 to the storage compartment 216. Apertures formed within the distal portion of the interface member 224 may place the wicking element 208 in fluid communication with vaporizable material 350 held in the storage reservoir 216. The vaporizable material 350 may thus be drawn towards the heating element assembly 210 by wicking element 208 or directly into the heating element assembly 210 without the wicking element 208 as is known to those of skill in the art.

When energized, the resistive heating element 264 may heat the heating element assembly 210 and this may cause the porous ceramic to draw vaporizable material drawn into the heating element assembly 210 for being heated by the resistive heating element 264. By heating the vaporizable material 350 to the predetermined vaporization temperature, a phyto material vapor 70 may be emitted into the heating chamber 226 and upon an inhalation from the inhalation aperture, the emitted vapor 70 may flow through the fluid conduit 204 having sidewalls defined by the interface member 224 out towards the cartridge proximal end from the inhalation aperture.

Conventional 510 cartridges 200 as are known to those of skill in the art may have two electrically insulated electrical, an outside of the 510 thread 299a as a ground electrical contact and the air conduit and power port 299b as a positive contact.

When the cartridge 200 is positioned within the cartridge receptacle 116, the vapor may then be inhaled by a user of vaporizer device 100 and when the heating element assembly is energized. The predetermined vaporization temperature may vary depending on user preference and/or the form of the vaporizable material. The vaporization device 100 may then be activated to vaporize the vaporizable liquid in the cartridge 200 and generate phyto material vapor. A user may then inhale the emitted vapor through inhalation aperture 112 to achieve therapeutic effects.

A user may then fully insert the removable cartridge assembly 200 within the cartridge receptacle 106 by sliding the cartridge into the cartridge receptacle and then rotating the cartridge to secure the threads or in some embodiments using a magnetic coupling comprising at least one magnet for securing the cartridge distal end within the cartridge receptacle. A sliding operation is illustrated in FIG. 2E.

Referring to FIG. 2B, the control circuit assembly 108 may be positioned within the interior device space 106. For instance, control circuit assembly 108 may be positioned within the interior device space 106 and proximate the cartridge receptacle 106.

Referring to FIG. 2C, the control circuit assembly 108 or control circuit assembly may be enclosed within the device body 102 and the control circuit assembly 108 may include a control circuit assembly 120, one or more wireless communication modules (122, 124, 126) such as Bluetooth 122, near-field communication (NFC) 124, and Wi-Fi module 126, and the energy storage module 128, such as one or more batteries. The control circuit assembly 120, Bluetooth module 122, NFC module 124, Wi-Fi module 126, and energy storage module 128 may all be mounted on, or supported by, the assembly support base 114. In some embodiments, the assembly support base 114 may include a motherboard that permits electrical communication between all components mounted thereon.

Energy storage module 128 may be electrically coupled to the control circuit assembly 120 and the one or more wireless modules. The control circuit assembly 120 may be electrically coupled to the wireless modules and may be configured to control the operation of the Bluetooth module 122, the NFC module 124 and the Wi-Fi Module 126. The wireless modules may allow firmware installed on vaporizer device 100, such as the control circuit assembly 120, to be updated remotely (e.g. from a central server or through a user application).

Control circuit assembly 120 may be configured to monitor and control various components of vaporization device 100. For example, control circuit assembly 120 may be used to monitor and control the flow of current from energy storage members 128 to the heating element assembly 210.

Control circuit assembly 120 may also be used to provide user interface functionality and user feedback, such as audio or visual outputs. The control circuit assembly 120 may also be used to control the operation of vaporization device 100, such as monitoring device activation and controlling operation of a heating assembly that is onboard vaporization device 100 (including heating elopement assembly provided within removable phyto material cartridges).

Control circuit assembly 120 may also monitor the state of various components of vaporization device 100, such as battery discharge levels, a fluid flow sensor 142 activity or other sensor signals, such as potentially a temperature sensor or a sensor used to measure current being provided to the heating element assembly or a gravity sensor, heating element temperature and so forth. Control circuit assembly 120 may also monitor one or more device sensors and feedback indicators, examples of which are described in further detail below.

In some embodiments, energy storage module 128 may be a rechargeable energy storage module, such as a battery or super-capacitor. Vaporization device 100 may include a power supply port (e.g. a USB-port 3232 or magnetic charging port or wireless charging port, such as Qi wireless charging standard) that allows the energy storage module 128 to be recharged. The energy storage module 128 may optionally be removable to allow it to be replaced.

In some embodiments, the vaporization device 100 may include a plurality of device status indicators, such as a plurality of LEDs 130 as shown in FIG. 2A. The status indicators may include various types of status indicators, such as auditory indicators, visual indicators, haptic feedback (e.g. a vibrating motor). The device status indicators may provide a user with information or feedback on various aspects of the vaporization device operation, such as remaining battery capacity, on/off status, mode of operation (e.g., high heat, medium heat, or low heat), temperature of a heating assembly, fill status of a cartridge, presence or absence of a cartridge in cartridge receptacle 116, whether to initiate an inhalation, whether to inhale deeper, whether to stop inhalation and so on.

For example, one or more indicator lights (e.g. Light-emitting diodes) may be provided on the vaporization device 100. The indicator lights may be electrically coupled to the control circuit assembly 120. Accordingly, the control circuit assembly 120 may control the operation of the indicator lights, as the plurality of LEDs 130. The indicator lights may be visible from the exterior of vaporizer device 100, to allow a user to easily identify the status of the vaporizer device 100. In the example shown, the indicator lights may include a plurality light emitting diodes (LEDs) 130.

The vaporizer device 100 may include the cover 144. The cover 144 may be secured to base 104 to enclose components of the vaporizer device 100.

As shown, the cover 144 may be secured to base 104 overlying the cartridge receptacle 106. The cover 144 may thus enclose the support member 114, and associated components mounted thereon. The cover is shown attached in FIG. 1A and removed in FIG. 1B.

Optionally, device cover 144 may be removably mounted to the body device 102. This may permit access to the control circuit assembly 108 for repairs and/or replacement. In other cases, the device cover 144 may be fixed to base 104 with the control circuit assembly 108.

Device cover 144 may or portion of the vaporizer housing or bas 104 may be manufactured of a non-conductive material and the device base 104 may be manufactured from a metal material, such as die casting. This may facilitate communication using the wireless modules disposed within the receptacle 106. In some embodiments, the device cover 144 may be from rubber or thermoplastic materials. The device cover 144 may be manufactured using material with a higher coefficient of friction than device base 104. This may facilitate attaching and removing the device cover 144 from base 104. The cover 144 may also provide a different tactile sense for a user gripping vaporizer device 100. Base 104 may be manufactured using a metallic material. For example, the base 104 may be manufacturing using a machining process, such as a Computer Numerical Control (CNC) machining process. In other cases, the base may be manufacturing using a metal injection molding (MIM) process or a die casting process. In general, however, the base 104 may be formed as a unitary base (i.e. base 104 may have a unitary construction). Alternative materials may also be used for the base 104. Ceramics, such as ceramics containing zirconium oxide, may be used to manufacture base 104. Alternatively, thermoplastic polymer materials may be used to manufacture base 104.

Referring to FIG. 2E and FIG. 2E, a cutaway view of the air intake manifold 110 is shown that may have a first manifold end 110A and a second manifold end 110B opposite the first manifold end 110B. In the example shown, the air intake manifold 110 may be mounted on the assembly support base 114.

Air intake manifold 110 may include a manifold fluid flow channel 136 defined therethrough. The manifold 110 may include at least one air input aperture 138, which may be referred to as an ambient air inlet or ambient air aperture that is exposed to an outside environment of the device 100 for facilitating incoming ambient airflow. The manifold 110 may also include a manifold outlet port 139 at the second manifold end 110B. The manifold outlet port 139 may be positioned facing the cartridge receptacle 116. The manifold fluid channel 136 may extend between the one or more ambient air inlets 138 or manifold inlet port and the manifold outlet port 139, defining a fluid passage between the ambient air inlet port and the cartridge receptacle 116.

In some embodiments one or more porous screens may be disposed within fluid channel 136, the porous screens may be configured to encourage laminar air flow in the ambient air entering fluid channel 136. The screen or screens may have pores of about 0.1 mm or 0.2 mm or 0.3 mm. The screens may also filter the ambient air to prevent dirt or debris from entering fluid channel 136 and may also provide for laminar flow into and through portions of the fluid flow channel 136. In other cases, a pressure drop element 1623 may be provided within the fluid channel 136 in the case of a differential pressure sensor being used as the fluid flow sensor 142.

Referring to FIGS. 2C, and 2D in some embodiments, the air intake manifold 110 may include the fluid flow sensor 142. The fluid flow sensor 142 may be configured to determine a volume or mass of ambient air 60 being drawn into the manifold fluid flow channel 136 and have its sensing ports fluidly coupled with the fluid flow channel 136. Optionally, an audio microphone may be positioned with the manifold fluid flow channel 136 to determine a volume or mass of airflow passing through the air intake manifold 110. In some embodiments a pressure sensor or a puff sensor may also be utilized to provide a binary indication of airflow through the fluid flow channel 136.

The fluid flow sensor 142 may be electrically coupled to the control circuit assembly 120. In some embodiments. The fluid flow sensor 142 may provide airflow signals to control circuit assembly 120. The control circuit assembly 120 may use the flow signals to determine the air flow through the air intake manifold 110 for controllable application of power to the heating element assembly 210. Based on detected airflow, the control circuit assembly 120 may perform various operations, such as activating/deactivating the heating assembly and/or adjusting a measured temperature of heating element assembly 210.

The cartridge may be inserted into the cartridge receptacle 106 until the distal end 200a of the cartridge 200 engages the air intake manifold 110 at the manifold outlet port 139 at the second manifold end 110B and the cartridge is secured by threaded engagement or magnetic engagement within the cartridge receptacle 106. The cartridge 200 may thus be releasably secured within the cartridge receptacle 106.

Referring to FIG. 1B, the cartridge 200 may have a cartridge port 167 proximate the distal end 200B that is for threading or magnetically engaging into a cartridge coupling port 1399 proximate the manifold outlet port 139 that may have a female 510 thread for engaging the cartridge port 167 having in some embodiments a male threaded end. The cartridge coupling port 1399 may provide an electrical coupling from the control circuit assembly 108 to the heating assembly of the cartridge 200. This electrical coupling may be for example a ground coupling disposed outwardly from a controlled power coupling that is used to controllably apply electrical energy to the heating assembly.

The cartridge port 167 may have two electrically insulated electrical contacts electrically coupled with the heating element assembly 210 with the fluid conduit propagating through a center thereof. The cartridge coupling port 1399 may be for electrically coupling to the heating element assembly 210 through first and second electrical connections, FIG. 2F, 299b and 299a.

When a user inhales from cartridge inhalation aperture 112, ambient air 60 (FIG. 1D) may be drawn from the external environment into the manifold fluid flow channel 136 via the at least one air input aperture 238. While being drawn by the user's inhalation through the fluid conduit 204, the ambient air 60 may mix with the vapor 70 emitted within the heating chamber conduit section prior to exiting the inhalation aperture 112.

Preferably, user inhalation and the vaporization of the vaporizable material 50 may be synchronized. In some cases, the control circuit assembly 108 may activate the heating element assembly 210 (or provide a signal to cartridge control circuit to activate the heating element assembly 210) in response to the fluid flow sensor 142 detecting ambient air passing through the air intake manifold 110.

Additionally, or alternatively, the plurality of LEDs 130 may indicate that the heating element assembly 210 is heated to the predetermined vaporization temperature. This may indicate that the vaporization device 100 is ready for a user inhalation. In other cases, alternative status indicators may be used. For instance, a vibration notification may be used to notify the user to initiate inhalation, to stop inhalation and/or to increase a depth of inhalation.

Using signals from the auditory sensor 143 or a puff sensor to activate the control circuit assembly 120 may allow the vaporization device 100 to conserve energy when the device 100 is not being used.

Referring to FIG. 2E in conjunction with FIG. 2C. In the example shown in FIG. 2E for the intake manifold 110 in a cutaway view for clarity, there may be an upstream input port 142a and there may be a downstream input port 142b in fluid communication with a manifold fluid flow path 136. A pressure sensing element may be disposed at the at least one of the upstream port 142a and downstream port 142b. Each pressure sensing element can determine an absolute pressure at the upstream port 142a and downstream port 142b and a single pressure sensing element can determine an absolute pressure at its respective port. A first absolute barometric pressure sensor 498 is fluidly coupled with its sensing port with the upstream port 142a and a second absolute barometric pressure sensor 499 is fluidly coupled with its sensing port with the downstream port 142b.

With reference to FIGs, 1E and 2E, in some embodiments the first absolute barometric pressure sensor 498 and the second absolute barometric pressure sensor 499 may be of the type BMP280 as manufactured by Bosch Sensortech. In some cases other pressure sensors may be used such as BME280 that also include an integrated environmental sensor with sensors for pressure, humidity and temperature in an 8-pin metal-lid in an approximately 2.5×2.5×0.93 mm³ LGA package, designed for low current consumption (ie 3.6 μA @1 Hz). Having an absolute pressure sensor also allows for pressure altitude determination whereby using pressure data from one of the sensors allows for an approximate pressure altitude of the vaporization device 400 to be known.

Having a possibility to read temperature allows for a temperature of incoming air to be known. In some embodiments one of the sensors may be an absolute pressure sensor and the other of the pressure sensors may also include absolute pressure and temperature and in some cases may include humidity sensing. Knowing temperature, absolute pressure and differential pressure allows for calibration with the differential pressure to obtain volumetric flow. In some embodiments, the differential pressure sensors are sealed to prevent air leakage around their housings.

FIG. 3A illustrates graphs of the first and second pressure signals 498a and 499a generated by the first and second absolute pressure sensors as well as a differential pressure signal 497 as determined by the control assembly. At a first portion of the graph there are shown three standard inhalations, 491, 492 and 493 (FIG. 3B) as well as a controlled inhalation 494 of 800 ml in about 6 seconds (as shown in FIG. 3E).

The first and second differential pressure sensors 498 and 499 output their respective first and second pressure signals in Pascals (Pa), 498a and 499a. An absolute offset between the first and second signals as first and second baseline levels 498b, 499b is shown in the graph of FIG. 3A and is due to absolute reading differences in the sensors and this may be calibrated through a lookup table. For example, the first baseline level 498b for the first differential pressure sensor 498 is about 29.8 kPa and the second baseline level 499b for the second differential pressure sensor 499 is about 29.9 kPa.

The first absolute barometric pressure sensor 498 and a second absolute barometric pressure sensor 499 provide the first and second pressure signals 498a and 499a. Each pressure signal is processed by a control circuit 420. Disposed between the upstream input port 442a and a downstream input port 442b there may be a pressure drop element 490, such as a raised protrusion or another form of obstruction that provides for a flow restriction of air flowing through the manifold fluid flow path 436.

In some embodiments when a single absolute barometric pressure sensor 498 is utilized, the baseline level may be obtained from the sensor as the first pressure signal and this baseline level 498b may be indicative of an absolute barometric pressure being sensed at, for example the upstream port 442a. The first pressure may be at an other than at the first baseline level 498c when air is flowing through the fluid flow path and the first absolute barometric pressure sensor 498 for providing the first pressure signal 498a having the first baseline level 498b provided to the control circuit when air is other than flowing through the fluid flow path.

In some embodiments when two absolute barometric pressure sensors are utilized, a second baseline level 499b may be obtained from the second sensor 499 as the second pressure signal 499a and this baseline level 499b may be indicative of an absolute barometric pressure being sensed at, for example the downstream port 442b. The first pressure may be at an other than at the first baseline level 498c when air is flowing through the fluid flow path and the first absolute barometric pressure sensor 498 for providing the first pressure signal 498a having the first baseline level 498b provided to the control circuit when air is other than flowing through the fluid flow path.

In some embodiments power from the control circuit may be applied to the heating assembly in dependence upon a difference between the first pressure signal and the second pressure signal. In some embodiments power from the control circuit is applied to the heating assembly in dependence upon a difference between the first pressure signal and the second pressure signal and a pulsewidth modulation profile of the power from the control circuit is applied to the heating assembly is varied in relation to the difference.

FIG. 3B, illustrates a mass airflow standard inhalation profile from an average user, for example inhalation 492, is similar to that shown in FIG. 3A where the differential pressure signals are shown as well as the difference between differential pressure signals. The inhalation volumes expressed in FIG. 3B, are full breath inhalations which are over two litres in volume and the graph illustrates the differential pressure for this inhalation.

FIG. 3C, illustrates a differential pressure signal of an inhalation of about 400 ml in about 3 seconds. FIG. 3D, illustrates the differential pressure signal of inhalation about 600 mL in about four seconds and FIG. 3E, illustrates the differential pressure inhalation of about 800 ml in about six seconds.

A difference between the barometric pressure sensors 498 and 499, which is a first absolute barometric pressure sensor 498 and a second absolute barometric pressure sensor 499 (resulting from the pressure drop within the fluid flow path 436 caused by the pressure drop element 490) may be used to determine the mass airflow or volumetric airflow and the airflow profile (change in airflow over time), whereby the airflow profile may be used to alter a heating power applied to the heating element assembly in accordance with embodiments of the invention. The differential pressure signal 497 may then be used to determine the volume or mass of air being drawn into vaporization device 400 over time to obtain an inhalation profile.

This differential pressure signal 497 may then be correlated using a lookup table with values providing a correlation between pressure difference as detected by the first absolute barometric pressure sensor 498 and a second absolute barometric pressure sensor 499 and mass air flow over time or volumetric airflow over time. In some cases when the single absolute pressure sensor is used, then a deviation of the sensor reading from the baseline reading may be used to approximate a mass airflow of air propagating through the air intake manifold as is shown in FIG. 3A for the levels 499c and 498c.

In some cases, the correlation between the mass air flow sensed and the volume of air entering the air intake manifold 410 may vary based on the temperature of the ambient air. The air intake manifold 410 may include an air temperature sensor or in some embodiments one of the pressure sensors includes a temperature sensor. The air temperature sensor can be configured to measure a temperature of air propagating in a non-bypass configuration between the between the upstream port 442a and downstream port 442b. The first and second barometric pressure sensors measure the differential pressure of air flowing through the manifold fluid flow path 436 in a non bypass configuration.

The barometric pressure sensors 498 and 499 may be electrically coupled to the control circuit 120. In some embodiments, the barometric pressure sensors 498 and 499 can be electrically coupled to the control circuit 120 through the assembly support base 114. The barometric pressure sensors 498 and 499 can provide flow signals to control circuit 120. The control circuit 120 may use the flow signals to determine the air flow through the air intake manifold 110. Based on the detected airflow, the control circuit 120 may perform various operations, such as activating/deactivating the heating assembly and/or adjusting a temperature of the heating assembly and or reading of the differential pressure profile over time and altering heating of the heating element.

A user may then fully insert the removable cartridge assembly 200 within the cartridge receptacle 116 by sliding the cartridge into the cartridge receptacle and then rotating the cartridge to secure the threads.

When energized, the heating element assembly 210 that may at least partially enclose the resistive wire 319 can emit heat to heat the porous ceramic that acts as a wick 208. The vaporizable material drawn into wick 208 can then be heated as well. By heating the vaporizable material 50 to a predetermined vaporization temperature, a phyto material vapor 70 can be emitted. The predetermined vaporization temperature may vary depending on user preference and/or the form of the vaporizable material.

The vapor can then pass through vapor aperture into the fluid conduit 204. The vapor can travel through the fluid conduit 204 towards the cartridge aperture 218. When the cartridge 200 is positioned within the cartridge receptacle 116, the vapor can then be inhaled by a user of vaporizer device 100 and when the heating element assembly is energized.

For example, vaporization device may store a calibration lookup table usable to correlate the voltage and current through the resistive heating element 264 with the temperature of heating element assembly 210 and in conjunction with the absolute pressure signals.

Power applied to the heating element may vary with battery voltage and may differ in its duration of application where for a battery that has a higher charge, a pulse-width modulation (PWM) profile is applied to heating element maybe a longer duration than when a battery is fully charged. In some embodiments, because the heating of the heating element takes place in such a short amount of time, in an order of about one two three seconds, it may be preferable to not sense a temperature of the heating element or a sensor resistance of the heating element wire, and it may be preferable to provide a lookup table that is predetermined for the application of power to the heating element. And some embodiments, a resistance of the heating element it may dictate the power profile as applied to the heating element. For a resistance of the heating element that is lower there may be a different PWM power profile applied to the heating element than for a higher resistance heating element.

In some embodiments, the air intake manifold 410 can include an auditory sensor 443 disposed proximate the air inlet 438. The auditory sensor 443 may be a microphone disposed facing the manifold fluid flow path 436 proximate ambient air inlet 438. The auditory sensor 443 may be used to detect air flow into the ambient air inlet 438. The auditory sensor 443 can output a volume signal to the control circuit 420 that can be used to determine whether ambient air 360 is being drawn into the air intake manifold 410. In some cases, the auditory sensor 443 can be configured with a volume threshold. When the volume threshold is reached, the auditory sensor 443 may transmit an air flow detection signal. This signal may be used (as an alternative to, or in combination with signals from mass airflow sensor 442) to wake the control circuit 420 from a low power or sleep mode. In some cases, the auditory sensor 443 may be mounted within the air intake manifold by an insulating material, such as rubber, to reduce false triggers. In some embodiments, at least one of the first or second barometric pressure sensors, is sampled at a predetermined frequency, and if a rise in pressure or falling pressure is detected, then the other of the first or second barometric pressure sensors is sampled by the control circuit to determine a pressure flow difference within the manifold fluid flow path 436.

Using signals from the airflow sensor 442 (generally representing the first absolute barometric pressure sensor 498 and the second absolute barometric pressure sensor 499) and/or auditory sensor 443 to activate the control circuit 420 may allow the vaporization device 400 to conserve energy when the device 400 is not being used. In some cases the first and second barometric sensors may be configured to operate semi-continuously (e.g. at 0.5 Hz, 1 Hz, 2 Hz) in a low power mode to measure a pressure differential between upstream port 442a and downstream port 442b.

For example, the fluid conduit 504 may have a cross-sectional area of about 4 mm2 or greater. In some cases, the cross-sectional area of the fluid conduit 504 may be about 5 mm2 (e.g. a width of about 5 mm and a height of about 1 mm). In some cases, the cross-sectional area of fluid conduit 504 may be about 6 mm2 (e.g. a width of about 6 mm and a height of about 1 mm).

Referring to the various embodiments descried herein, enabling a user to perform a deep inhalation (e.g. an inhalation that approaches a lung tidal volume such as 0.3 L, 0.4 L, or 0.5 L), rather than merely a puff (e.g. 0.1 L, 0.2. L or less), increases the likelihood of the aerosolized vaporizable material in the emitted vapor penetrating more deeply into the user's lungs. This may allow for improved absorption by the user's alveoli for aromatherapy benefits.

As used herein, the wording “and/or” is intended to represent an inclusive—or. That is, “X and/or Y” is intended to mean X or Y or both, for example. As a further example, “X, Y, and/or Z” is intended to mean X or Y or Z or any combination thereof.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What I claim is:

1. A vaporizer device comprising:
a vaporizer body comprising:
an elongated base extending from a first end to a second end, the elongated base including a pair of opposed sidewalls extending between the first end and the second end and a second end wall at the second end;
a mouthpiece formed at the second end of the base, the mouthpiece comprising an inhalation aperture through the second end wall;
an air intake manifold mounted to the base, the air intake manifold having a first manifold end and a second manifold end, the air intake manifold comprising an ambient air input port disposed between the first manifold end and the second manifold end, the ambient air input port being exposed to an external environment
a fluid flow path formed between the first manifold end and the second manifold end the fluid flow path comprising an upstream input port in fluid communication with a first absolute barometric pressure sensor and a downstream input port in fluid communication with a second absolute barometric pressure sensor,
each of the pressure sensors for providing of a respective pressure signal to a control circuit, the control circuit for calculating a differential pressure signal from the respective pressure signals;
an elongated storage compartment, the storage compartment being configured to store a vaporizable material, the storage compartment comprising an inner storage volume wherein the vaporizable material is storable in the inner storage volume, wherein the inner storage volume is at least partially enclosed by a storage volume housing having a first end and a second end opposite the first end;
a heating assembly disposed at the storage volume first end and electrically coupled with the control circuit, the heating assembly comprising a heating element and a wicking element, wherein the heating element thermally coupled to the wicking element, and wherein the wicking element is in fluid communication with the inner storage volume; and
a fluid conduit extending through the storage volume housing, the fluid conduit having a fluid conduit inlet at the storage volume first end and a fluid conduit outlet at the storage volume second end, wherein the fluid conduit is in fluid communication with the wicking element,
wherein when the inner storage volume is at least partially enclosed by the storage volume housing, the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing through the heating assembly whereby vaporized material is inhalable through the inhalation aperture and power from the control circuit is applied to the heating assembly in dependence upon the differential pressure signal.

2. A vaporizer device according to claim 1 wherein the respective pressure signals comprise first and second pressure signals and wherein the first absolute barometric pressure sensor and the second absolute barometric pressure sensor generate a first baseline signal and a second baseline signal when air is other than propagating through the fluid flow path.

3. A vaporizer device according to claim 1 wherein the respective pressure signals comprise first and second pressure signals and wherein the first absolute barometric pressure sensor and the second absolute barometric pressure sensor generate other than a first baseline signal and other than a second baseline signal when air is propagating through the fluid flow path.

4. A vaporizer device according to claim 3 wherein the first and second baseline signals are at other than a same level when air is other than propagating through the fluid flow path.

5. A vaporizer device according to claim 1 wherein the storage volume housing comprises a cartridge housing and the cartridge housing comprises a first cartridge end as the storage volume first end and a second cartridge end as the storage volume second end, wherein the cartridge housing is releasably mounted at least partially within the vaporizer body where the first cartridge end is releasably coupled with the fluid conduit and the electrically coupled with the control circuit.

6. A vaporizer device according to claim 1 wherein the fluid flow path formed between the first manifold end and the second manifold end the fluid flow path comprising a pressure drop element disposed within the fluid flow path between the upstream input port and the downstream input for creating a flow restriction of air flowing through the manifold fluid flow path.

7. A vaporizer device comprising:
a vaporizer body comprising:
an elongated base extending from a first end to a second end, the elongated base including a pair of opposed sidewalls extending between the first end and the second end and a second end wall at the second end;
a mouthpiece formed at the second end of the base, the mouthpiece comprising an inhalation aperture through the second end wall;
an air intake manifold mounted to the base, the air intake manifold having a first manifold end and a second manifold end, the air intake manifold comprising an ambient air input port disposed between the first manifold end and the second manifold end, the ambient air input port being exposed to an external environment and formed between the first end and the second end;
a fluid flow path formed between the first manifold end and the second manifold end the fluid flow path comprising an upstream input port in fluid communication with a first absolute barometric pressure sensor, the first absolute barometric pressure sensor for providing of a first pressure signal to a control circuit, the first pressure signal being at an other than a first baseline level when air is flowing through the fluid flow path and the first absolute barometric pressure sensor for providing the first pressure signal having the first baseline level to the control circuit when air is other than flowing through the fluid flow path;
an elongated storage compartment, the storage compartment being configured to store a vaporizable material, the storage compartment comprising an inner storage volume wherein the vaporizable material is storable in the inner storage volume, wherein the inner storage volume is enclosed by the elongated base and the elongated storage compartment is at least partially enclosed within the vaporized body;
a heating assembly electrically coupled with the control circuit, the heating assembly comprising a heating element and a wicking element, wherein the heating element thermally coupled to the wicking element, and wherein the wicking element is in fluid communication with the inner storage volume; and
a fluid conduit extending through a portion of the elongated base, the fluid conduit having a fluid conduit inlet and a fluid conduit outlet, wherein the fluid conduit is in fluid communication with the wicking element,
the fluid conduit inlet is fluidly connected to the air intake manifold and the fluid conduit outlet is fluidly connected to the mouthpiece, and a fluid flow passage is defined between the ambient air input port and the inhalation aperture, the fluid flow passage passing through the heating assembly whereby vaporized material is inhalable through the inhalation aperture and power from the control circuit is applied to the heating assembly in dependence upon the first pressure signal being other than at the baseline level.

8. A vaporizer device according to claim 7 wherein the fluid flow path additionally comprising an downstream input port in fluid communication with a second absolute barometric pressure sensor, the second absolute barometric pressure sensor for providing of a second pressure signal to a control circuit, the second pressure signal being at an other than second baseline level when air is flowing through the fluid flow path and the second absolute barometric pressure sensor for providing the second pressure signal having the second baseline level to the control circuit when air is other than flowing through the fluid flow path, wherein power from the control circuit is applied to the heating assembly in dependence upon a difference between the first pressure signal and the second pressure signal.

9. A vaporizer device according to claim 7 wherein the fluid flow path additionally comprising an downstream input port in fluid communication with a second absolute barometric pressure sensor, the second absolute barometric pressure sensor pressure sensors for providing of a second pressure signal to a control circuit, the second pressure signal being at an other than second baseline level when air is flowing through the fluid flow path and the second absolute barometric pressure sensor for providing the second pressure signal having the second baseline level to the control circuit when air is other than flowing through the fluid flow path, wherein power from the control circuit is applied to the heating assembly in dependence upon a difference between the first pressure signal and the second pressure signal and a pulsewidth modulation profile of the power from the control circuit is applied to the heating assembly is varied in relation to the difference.

10. A vaporizer device according to claim 8 wherein at least one of the second absolute barometric pressure sensor and the first absolute barometric pressure sensor comprises a temperature sensor.

11. A vaporizer device according to claim 9 wherein the fluid flow path formed between the first manifold end and the second manifold end the fluid flow path comprising a pressure drop element disposed within the fluid flow path between the upstream input port and the downstream input for creating a flow restriction of air flowing through the manifold fluid flow path for creating the signal difference.

12. A vaporizer device comprising:
a cartridge comprising:
a cartridge housing extending from a distal end of the cartridge to a proximal end of the cartridge;
an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing;
a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and
a fluid conduit extending through the cartridge housing from a conduit inlet at the distal end to a conduit outlet at the proximal end, wherein the fluid conduit is fluidly connected to the heating element assembly, the fluid conduit passes through a center of the heating assembly from the distal end to the proximal end;
an inhalation aperture formed at the proximal end of the fluid conduit;
a cartridge port having two electrically insulated electrical contacts electrically coupled with the heating element assembly with the fluid conduit propagating through a center thereof;
a device body comprising:
a cartridge coupling port for electrically coupling to the heating element assembly through first and second electrical contacts and fluidly coupling of the manifold outlet with the fluid conduit distal end;
a fluid flow path formed between manifold outlet and an ambient air inlet port, the fluid flow path comprising an upstream input port in fluid communication with a first absolute barometric pressure sensor coupled with a control circuit assembly electrically coupled with the cartridge coupling port, the first absolute barometric pressure sensor for providing of a first pressure signal to a control circuit, the first pressure signal being at an other than a first baseline level when air is flowing through the fluid flow path and the first absolute barometric pressure sensor for providing the first pressure signal having the first baseline level to the control circuit when air is other than flowing through the fluid flow path, the control circuit assembly for controllably providing of pulse width modulated electrical power to the heating assembly in dependence upon the first pressure signal being at an other than a first baseline level and for other than controllably providing of pulse width modulated electrical power to the heating assembly when the first pressure signal is at the first baseline level,
wherein when the cartridge is inserted into the vaporization device the cartridge coupling port is coupled with the cartridge port which electrically couples the heating assembly with the control circuit assembly,
wherein the storage compartment surrounds the heating assembly and the fluid conduit; and
wherein the fluid conduit extends along the entire length of the elongated storage compartment from the distal end to the proximal end.

13. A vaporizer device according to claim 12 wherein the fluid flow path additionally comprising an downstream input port in fluid communication with a second absolute barometric pressure sensor, the second absolute barometric pressure sensor pressure sensors for providing of a second pressure signal to the control circuit assembly, the second pressure signal being at an other than second baseline level when air is flowing through the fluid flow path and the second absolute barometric pressure sensor for providing the second pressure signal having the second baseline level to the control circuit when air is other than flowing through the fluid flow path, wherein power from the control circuit is applied to the heating assembly in dependence upon a difference between the first pressure signal and the second pressure signal and a pulsewidth modulation profile of the power from the control circuit is applied to the heating assembly is varied in relation to the difference.

14. A vaporizer device according to claim 12 wherein the fluid flow path formed between the first manifold end and the second manifold end the fluid flow path comprising a pressure drop element disposed within the fluid flow path between the upstream input port and the downstream input for creating a flow restriction of air flowing through the manifold fluid flow path for creating the signal difference.

15. A vaporizer device according to claim 12 wherein the fluid flow path formed between the first manifold end and the second manifold end and the storage compartment, heating assembly are concentrically disposed.
